(19) United States
(12) Patent Application Publication
(10) Pub. No.: US 2018/0280175 A1
(43) Pub. Date: Oct. 4, 2018

(54) ASSISTANCE SYSTEM FOR COGNITIVELY IMPAIRED PERSONS

(71) Applicant: KONINKLIJKE PHILIPS N.V., EINDHOVEN (NL)

(72) Inventors: Charles Frederik Sio, Eindhoven (NL); Maarten Petrus Joseph Kuenen, Veldhoven (NL); Marc Andre de Samber, Lommel (BE); Murtaza Bulut, Eindhoven (NL)

(73) Assignee: KONINKLIJKE PHILIPS N.V., Eindhoven (NL)

(21) Appl. No.: 15/763,877

(22) PCT Filed: Sep. 28, 2016
(86) PCT No.: PCT/EP2016/073045

§ 371 Date: Mar. 28, 2018

(30) Foreign Application Priority Data
Sep. 30, 2015 (EP) 15187632.3

(51) Int. Cl. A61F 4/00; A61B 5/16; A61B 5/1171; A61B 5/11; G05B 19/042

(52) U.S. Cl. CPC A61F 4/00; A61B 5/165; A61B 5/167; A61B 5/02438; A61B 5/1116; A61B 5/163; G05B 19/0426; A61B 5/1176

(57) ABSTRACT

A device for providing assistance to a person comprises a data input for obtaining person related data representing data obtained by monitoring the person, a data processing unit for processing the obtained person related data to determine the person's mental state and/or cognitive level from the obtained person related data, to detect if the person has limited or no ability to perform a desired task and to generate assistance data in case of detection that the person has limited or no ability to perform the desired task and/or if the person's current mental state and/or cognitive level is below a threshold or out of a distribution or value range required to perform the desired task, said assistance data representing data directed to supporting the person in performing the desired task by adapting the number of functions of an appliance accessible and controllable by the person according to the person's current mental state and/or cognitive level, and a data output for providing said assistance data for supporting the person in performing the desired task.

### FIELD OF THE INVENTION

The present invention relates to a device for providing assistance to a person, in particular to a person whose mental condition is not sufficient to carry out tasks, e.g. connected to operating a household appliance. The invention further relates to a system comprising said device in communication with a monitoring device collecting personal data and a user device in communication with the user.

### BACKGROUND OF THE INVENTION

Cognitive impaired persons experience difficulties in using devices and appliances to perform activities of daily life (ADLs). This causes several problems, of which the following belong to the most wearing ones: stress in caregivers who are responsible for the welfare of the cognitively impaired person at all times, which has been shown to have an adverse effect on the caregiver's health and well-being, and reduction of the autonomy of the cognitively impaired person, when the caregiver takes over tasks from the cognitively impaired person. The latter problem is highlighted by the high prevalence of depression and related conditions in Alzheimer's Disease (AD) and other dementia patients.

Current solutions are either to prevent use of devices and appliances by the cognitive impaired person, or to limit the user interface and/or functionality. These solutions are semi-permanent, and therefore the caregiver and others in the household will be hampered in the normal use of the device, leading to frustration and a low level of adoption. More fundamentally, current solutions are not specifically tailored to the cognitive skill level of the impaired person.

People having symptoms of Alzheimer's Disease (AD) or dementia (because of other causes than AD) suffer from reduced skills. This has major implications in daily life. The main limitations of an AD patient are a decline in cognitive and hence also physical abilities, requiring guidance and support, and a decline which is slowly progressive, requiring long-term adaptations in levels of guidance and support. The skills may vary over time, as do the mood and personality of the cognitively impaired person, leading to ‘good’ and ‘bad’ days, thus requiring, in addition, also short-term adaptations.

Unsupervised, a person with declining cognitive and physical abilities can still carry out activities of daily life, but at the risk of damage to the house and devices, frustration at their own lack of ability which may lead to outbursts of anxiety and/or aggressiveness and personal injury. These risks can be mitigated by preventing the person from performing the tasks, but this is known to cause frustration and anxiety making the person hard to handle by caregivers, and have a negative impact on the rate of cognitive decline. Keeping the patients as active as possible increases the quality of life of the patient and decreases the burden on caregivers.

These patients often enjoy doing practical activities in their own environment. For example, preparing meals is for an (earlier phase) AD patient a very useful, rewarding and enjoying activity. In contrast, not being able or allowed to prepare their own food is a serious limitation of their independence. Such or comparable activities in the house become largely jeopardized by the mentioned symptoms. As a result the activities can lead to e.g. an improper result (e.g. bad quality of prepared food), an incomplete result and/or unsatisfactory experience by the patient. But even worse, these can also lead to dangerous and even life threatening situations when e.g. kitchen appliances are used incorrectly.

US 2014/0007009 A1 discloses a mobile video-based therapy which uses a portable therapy device that includes a camera, a therapy application database, a processor, and a display. The camera is configured to generate images of a user, and the therapy application database is configured to store therapy applications. The processor is configured to select, from the therapy application database, a therapy application appropriate for assisting in physical or cognitive rehabilitation or therapy of the user, to invoke the therapy application, to recognize a gesture of the user from the generated images, and to control the invoked therapy application based on the recognized gesture. The display is configured to display an output of the controlled therapy applications.

US 2004/0030531 A1 refers to an automated system and method for monitoring and supporting and actor in an environment, such as a daily living environment. The system includes at least one sensor, at least one effector and a controller adapted to provide monitoring, situation assessment, response planning, and plan execution functions. In one preferred embodiment, the controller provides a layered architecture allowing multiple modules to interact and perform the desired monitoring and support functions.

### SUMMARY OF THE INVENTION

It is an object of the present invention to provide an enhanced device, system and method for providing assistance to a person of limited ability to perform certain tasks, in particular within the person's home environment, to facilitate and support the person's abilities for self-care with regard to daily life.

In a first aspect of the present invention a device for providing assistance to a person is presented, said device comprising a data input for obtaining person related data representing data obtained by monitoring the person, a data processing unit for processing the obtained person related data to determine the person's mental state and/or cognitive level from the obtained person related data, to detect if the person has limited or no ability to perform a desired task, and to generate assistance data in case of detection that the person has limited or no ability to perform the desired task and/or if the person's current mental state and/or cognitive level is below a threshold or out of a distribution or value required to perform the desired task, said assistance data representing data directed to supporting the person in performing the desired task, wherein said assistance data are used to adapt the functionality of an appliance by adapting the number of functions accessible and controllable by the person according to the person's current mental state and/or cognitive level, and a data output for providing said assistance data for supporting the person in performing the desired task.

In a further aspect of the present invention a system is presented which comprises the proposed device as well as monitoring means for monitoring the person and generating person related data, and a user device for obtaining and/or using said assistance data to support the person in performing the desired task.

In a still further aspect of the present invention a method for providing assistance to a person is presented.

In yet further aspects of the present invention, there are provided a computer program which comprises program code means for causing a computer to perform the steps of the method disclosed herein when said computer program is carried out on a computer as well as a non-transitory computer-readable recording medium that stores therein a computer program product, which, when executed by a processor, causes the method disclosed herein to be performed.

Preferred embodiments of the invention are defined in the dependent claims. It shall be understood that the claimed system, method, computer program and medium have similar and/or identical preferred embodiments as the claimed device, in particular as defined in the dependent claims and as disclosed herein.

The present invention is based on the idea to provide assistance autonomously or in controlled fashion adapting according to the skill level of the cognitively impaired person (also called user or patient herein), e.g. to provide adaptive interfaces or guidance systems on e.g. kitchen appliances that autonomously or in controlled fashion adapt according to the skill level of the person. The adaptation is preferably effectuated in real time, taking into account momentary person characteristics. The level of support or guidance depends on the apparent instantaneous skill for the currently performed task. The invention thus e.g. allows the use of the various appliances (which includes any household device, e.g. in the kitchen, or any device for home healthcare, e.g. a blood pressure measurement device) by various persons with various skill levels even if the individual's skill level is not constant. The proposed device, system and method thus enable a person to remain as independent and autonomous as possible regardless of fluctuations in performance and mental state. Daily tasks can be performed with an optimized amount of assistance and security for the person. Frustration, anxiety and inactivity can be prevented and thus the progression of dementia and depressions slowed down. Next to providing safety to the cognitively impaired person when not under direct supervision of a caregiver, it also provides assistance to reach the desired end result unless a dangerous situation is imminent.

According to an embodiment, the proposed device further comprises a recognition unit for recognizing the person's identity, wherein the data processing unit is configured to take the person's identity into account in the generation of the assistance data. An appliance thus can be set in correct functionality from the beginning without a start-up delay and resulting in an uncertainty of the person.

According to another advantageous embodiment, the assistance data are control data inducing the start, execution and/or end of a task on an appliance, the assistance data being transmitted to the appliance on which the task is being carried out when the data processing unit has detected that the person's ability to perform the task is below the threshold or out of the distribution or value range required to perform the task. In this way the danger of critical situations due to wrong actions of the person can be minimized. A refinement in the assistance level can be made by having multiple levels of state of the patient.

According to yet another advantageous embodiment, the assistance data include instructions how to carry out a task on an appliance, the assistance data being communicated to the person carrying out the task depending on the person's current mental state and/or cognitive level when the data processing unit has detected that the person's ability to perform the task is below the threshold or out of the distribution or value range required to perform the task. This allows the person the largest possible extent of self-reliance when performing the task on the appliance with the resulting sense of achievement.

According to another preferred embodiment, the person related data are obtained by monitoring a person by way of monitoring means, the person related data being one or more of: heart rate, pulse, blood pressure, sweat, skin and/or core body temperature, facial expression, gaze track, pupil size, posture, motion, trembling, repetitive movements, tics, twitches, voice, tactile abilities, amount of reaction to outer stimuli, reaction time to outer stimuli. Such parameters provide a wide variety of possibilities to check a person's physical and mental condition and optimize the assistance accordingly.

According to another embodiment, the data processing unit is configured to compare current person related data indicating the mental state and/or cognitive level to predetermined person related data and/or person related data from previous monitoring of the person and/or threshold values, distributions or other comparative values assigned to tasks and generating assistance data depending on the result of the comparison. Thus, the database is also suitable to be accessed by other appliances and immediately adapt the functionality of each appliance to the mental state and/or cognitive level of the person.

Advantageously, the data processing unit is configured to adjust the assistance data in dependence of the performance of the person in carrying out the task when the threshold value, the distribution range or other comparative value required for the task is exceeded or not accomplished. The varying performance over time and the increase or decrease in performance can be balanced in this way. In another advantageous embodiment, not only the performance of the task is taken into account for adjustment of the assistance, but also the monitoring data itself. Thus, monitoring of the effects of the interaction with the device on the cognitively impaired person is used for direct input to the adjustment of the assistance data.

According to an embodiment, the device further comprises a data storage to store the person related data, data obtained from the data input, the assistance data, threshold data, distribution or other value range data and/or comparative data derived from previous monitoring of the person. This is advantageous since the time to adapt the functionality of an appliance or to provide assistance data can be kept short with immediate readiness of the appliance.

The proposed system can be extended to the complete environment of a cognitively impaired person, for example to the whole flat or house. Caregivers being present in the house do not necessarily have to be present all the time in the same room as the person and still be in control of the situation. Persons can use the full bandwidth of the household or personal care appliance present in their environment.

In an embodiment of the system, the monitoring unit comprises one or more of a tactile recognition device, in particular a fingerprint scanner, a device for recognition and/or analysis of voice or speech characteristics, preferably a microphone, a facial recognition or facial expression device, a device for gaze tracking, a device for posture recognition or analysis, preferably a camera, a device for monitoring vital functions, preferably a pulse and/or heart rate and/or blood pressure and/or skin/body core temperature monitor, a device for detecting amount of response and reaction time to outer stimuli, in particular a timer, or an device for reading a personal identification tag. The wide variety of means provides solutions for any environment and for many different settings in which the person may live.

Preferably, the user device is configured to communicate instructions to the person carrying out a task on an appliance depending on the assistance data generated by the data processing unit when the data processing unit has detected that the person's ability to perform the task is below the threshold or out of the distribution or value range required to perform the task. Further, the user device may be configured to carry out a task on an appliance automatically when the data processing unit has detected that the person's ability to perform the task is below the threshold or out of the distribution or value range required to perform the task.

Still further, in an embodiment the system is configured to constantly collect data from the monitoring unit, to adjust the assistance data depending on the person's current mental state and/or cognitive level derived from said monitoring data by evaluation of the ability of the person to perform the task and to assign or revoke control to the person to perform the task based on said person's current mental state and/or cognitive level.

According to an advantageous embodiment of the system, the user device and/or interface is adaptable to the person's current mental state and/or cognitive level, in particular a touchscreen with a variable number of buttons displayed depending on the user, a control panel in which physical buttons are sunk or blended and only recognizable when the functionality of the control panel is adapted accordingly, or a control panel which is partly covered and only uncovered when the functionality of the control panel is adapted accordingly. The risk of malfunctions, of frustrating failure to comply with a task or of uncertainness and resulting stress and rising anxiety are thus minimized.

In an example of a device for providing assistance to a person, the device comprises: i) a data input for obtaining person related data representing data obtained by monitoring the person, ii) a data processing unit for processing the obtained person related data to: a) determine the person's mental state and/or cognitive level from the obtained person related data, b) detect if the person has limited or no ability to perform a desired task, and c) generate assistance data in case of detection that the person has limited or no ability to perform the desired task and/or if the person's current mental state and/or cognitive level is below a threshold or out of a distribution or value range required to perform the desired task, said assistance data representing data directed to supporting the person in performing the desired task, and iii) a data output (4) for providing said assistance data for supporting the person in performing the desired task.

In an example of a method for providing assistance to a person, the method comprises: i) obtaining person related data representing data obtained by monitoring the person, ii) processing the obtained person related data to: a) determine the person's mental state and/or cognitive level from the obtained person related data, b)detect if the person has limited or no ability to perform a desired task, and c) generate assistance data in case of detection that the person has limited or no ability to perform the desired task and/or if the person's current mental state and/or cognitive level is below a threshold or out of a distribution or value range required to perform the desired task, said assistance data representing data directed to supporting the person in performing the desired task, and iii) providing said assistance data for supporting the person in performing the desired task.

### BRIEF DESCRIPTION OF THE DRAWINGS

These and other aspects of the invention will be apparent from and elucidated with reference to the embodiment(s) described hereinafter. In the following drawings

FIG. 1 shows a schematic diagram of an embodiment of a device and a system according to the present invention,

FIG. 2 shows a diagram illustrating the tasks connected to an exemplary process to be carried out by an appliance suitable for use in the proposed system,

FIG. 3 shows a schematic diagram of an exemplary smart cooker according to the present invention,

FIGS. 4A and 4B show an embodiment of a TV remote control with adapted functionality for a person with decreased mental abilities and a caregiver, and

FIGS. 5A and 5B show an embodiment of a blood pressure gauge with adapted functionality for a cognitively impaired person.

### DETAILED DESCRIPTION OF THE INVENTION

The present invention proposes adaptive functionality, assistance and guidance systems on e.g. kitchen appliances, personal care appliances and the like that autonomously or in controlled manner adapt their functionality according to the skill level of the user. This adaptation is performed in real time, taking into account user characteristics and assessment of the previous user actions. The level of support/guidance depends on the apparent instantaneous skill for the currently performed subtask. As a result, the level of guidance may vary within a single task or activity, in order to maximize correct performance of the task/activity with a level of guidance/support that is precisely tailored to the specific user's needs and capabilities at this specific time. The present invention allows the use of the various appliances, e.g. in the kitchen or the bathroom, by various users with various skill levels even if the individual's skill level is not constant. No guidance by a caregiver (higher skilled user) would be required.

Adaptations might be e.g. easing a cooking process by taking over certain actions, giving guidance or reminders to the user at multiple levels of complexity, taking overrule actions to prevent mistakes or dangerous conditions, or escalating options to warn either the user or nearby higher skilled users like caregivers.

The present invention e.g. envisions appliances that have the functionalities user recognition, user monitoring, activity monitoring, data processing and adaptation of the functionality of the appliance. Preferably, this may be used in a connected system. Ideally, the appliance is equipped with an adaptive control panel which identifies the user and presents the user with the appropriate user interface and functionality of the appliance or device.

The present invention will now be described in more detail with reference to the drawings.

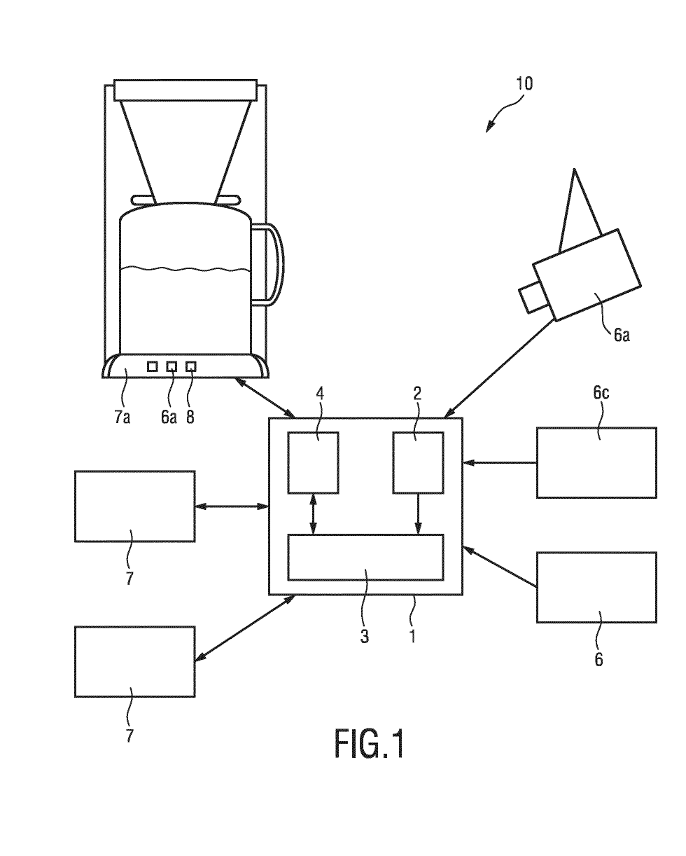

In FIG. 1, a schematic diagram of an exemplary embodiment of a device 1 and of a system 10 according for providing assistance data and respectively adapted functionality according to the present invention are shown. The device 1 comprises a data input 2, a data processing unit 3 and a data output 4.

The data input 2, e.g. a data interface, obtains person related data representing data obtained by monitoring the person, e.g. from a monitoring unit 6, such as a camera 6a, and optionally from an appliance 7, such as a kitchen device (e.g. a coffee maker 7a). The data processing unit 3, e.g. a processor or computer, processes the obtained person related data to determine the person's mental state and/or cognitive level from the obtained person related data, to detect if the person has limited or no ability to perform a desired task. Further, it generates assistance data in case of detection that the person has limited or no ability to perform the desired task and/or if the person's current mental state and/or cognitive level is below a threshold or out of a distribution or value range required to perform the desired task. Said assistance data represent data directed to supporting the person in performing the desired task. The data output 4, e.g. a user interface, such as a display or loudspeaker, provides said assistance data for supporting the person in performing the desired task. In an embodiment, the data output 4 conveys the generated assistance data to the at least one appliance 7 to assist a person in operating the appliance 7 or to adapt the functionality of the appliance 7 in accordance with the data received from the recognition and/or monitoring device 6 or from the appliance itself. Preferably, the components of the system 10 are in some way interconnected to form a connected system 10.

In the following, the elements of system 10 are described in more detail making reference to exemplary household or personal care appliances 7, such as a coffee maker 7a. Coffee makers are widespread appliances which come in various forms and technical embodiments with a wide variety of functions. For a person suffering from decreasing mental abilities the process of making coffee thus can become an unresolvable task which cannot be carried out any more due to the complexity of the stages of preparation. As mentioned above, this results in frustration of the patient or even to dangerous situations due to appliances 7 which are not operated appropriately.

Thus, the present invention seeks to facilitate the correct operation of the appliance 7 and assist the person if the task can only partially or not be executed at all by automatic and controlled adaptation of the guidance and functionality of the appliance 7 according the skill level of the user.

In the exemplary embodiment of a coffee maker 7a, user recognition may be done by a fingerprint scanner 6b, for example on the on/off button of the coffee maker 7a. This can also “awaken” other elements of a user interface or control panel 8. Other means for user recognition may include a microphone 6c for voice recognition, a camera 6a for facial recognition or posture recognition, or an identifier on the user such as a Wi-Fi signal from a smart watch, or an RFID tag on a pendant. As in most homes and most institutions the number of possible users will be limited, basic (i.e. cheap) devices and recognition software may suffice to distinguish the users.

The adaptable user interface or control panel 8 can e.g. be a touchscreen with different buttons to be displayed, or a control panel in which physical buttons are sunk/blended in the panel and are not recognized as button unless they are activated, or a control panel of which part is covered and to be uncovered if a user other than the cognitively impaired person is identified, etc. Another preferred option involves adaptation of the functionality, but not the interface itself, in a subtle manner to the detected cognitive skill level of the user.

User monitoring may be carried out by one or more of the components 6 in FIG. 1 which can be adapted to perform user recognition functions as well as user monitoring functions. For example, the camera 6a can on the one hand be used to recognize the user who is entering the room or starting to handle an appliance 7 and on the other hand monitor the user's performance during the operation of the appliance 7.

User characteristics to monitor comprise e.g. anxiety, overall cognitive ability and apparent task-specific understanding. This can be accomplished by monitoring parameters such as vital signs (heart rate and galvanic response), facial expression overall and in response to the offered help/guidance, voice recognition, general speech features (e.g. pitch, voice strength, speech hesitation), gaze tracking, and response time and response type to offered help/guidance. Sensors may include unobtrusive body-worn sensors such as smart watch, cameras on the device and throughout the room, etc.

Activity monitoring may be done by the same components 6 in FIG. 1, but also via switches in the appliances 7 or the environment, e.g. door open detector in cabinets, on-off switches of appliances 7, flow meter in a faucet etc. In this regard, features important to determine usage characteristics may e.g. comprise timing of user interaction, specific user actions as well as their order with respect to each other (in time). Detection of erroneous usage, e.g. wrong, insufficient or aborted input by the user, such as leaving the refrigerator open for a prolonged period as well as repetitions of the same user interaction can be monitored, too.

Data processing is performed by the data processing unit 3 in the device 1. Alternatively, a cloud-based control would be possible. Based on the detected user, the user's monitored actions, behaviors and activity, the device 1 determines its response, i.e., its output/feedback to the user.

The feedback in form of assistance data or adapted functionality of the appliance 7 is conveyed to the data output 4 and delivered to e.g. a screen with written instructions or to a microphone for verbal instructions. When the system 10 detects that the user's ability to perform certain tasks is e.g. below a threshold value or a value distribution assigned to the respective task, (partial) automation of a number of actions, like activating functions of appliances 7, powering down of appliances 7, alerting a caregiver etc. can be initiated.

The threshold which is assigned to the respective task can be set by various methods. In a very basic embodiment, the threshold can be derived from databases containing population statistics and stored in the system 10 e.g. as a table containing threshold values for each task. The threshold value can be pre-programmed as factory settings and then adjusted to the individual patient. Another option is the initialization of the threshold values for a specific cognitively impaired person upon first-time usage of the appliance 7 to set a baseline. Preferably, since especially dementia patients may have quite different cognitive impairments, the threshold values would be personalized by machine learning techniques that interactively personalize the threshold values based on the previous usage of the appliance 7 and on the outcomes of the usage, e.g. by analyzing whether the initially provided guidance was sufficient or additional guidance or caregiver action was needed. Alternatively, the threshold values can be determined and adjusted by the caregiver upon observation of the cognitively impaired person.

These considerations also apply to distribution ranges and other comparative value ranges which can be derived from statistics or pre-use in a similar manner. If e.g. a certain task requires an average time to finish, a time range can be specified during which a task can be performed faster or slower but not deviating from the average more than a specified amount. This is applicable, when there is past data, and when it is known how the past data will change with mental degradations. For example, it could be expected that the operation time of the appliance will increase with mental degradation.

Preferably, not only the performance of the task by the cognitively impaired person is taken into account for adjustment of the assistance, but also the monitoring data themselves. Thus, monitoring of the effects of the interaction with the appliance 7 on the cognitively impaired person is used for direct input to the adjustment of the assistance data. E.g. stress, anxiety, uncertainty, hesitation, anger or frustration, which can be monitored directly via one of the components 6 mentioned above, indicate that the currently provided guidance is not sufficient. The monitored values can then be used alongside the performance values to refine adjustment of the assistance data to the necessary level in a highly efficient manner. Calmness, purposefulness, with a bit of impatience indicate that the guidance is of sufficient detail and may be set a level up thus preventing frustration by too much assistance which is not necessary in this moment.

In a connected system 10, the information gathered by a number of recognition and/or monitoring devices 6 can be integrated to generate a more precise user guidance. This is especially valuable in tasks that require or benefit from the use of a (large) number of appliances 7 which are suitable to be connected to the system.

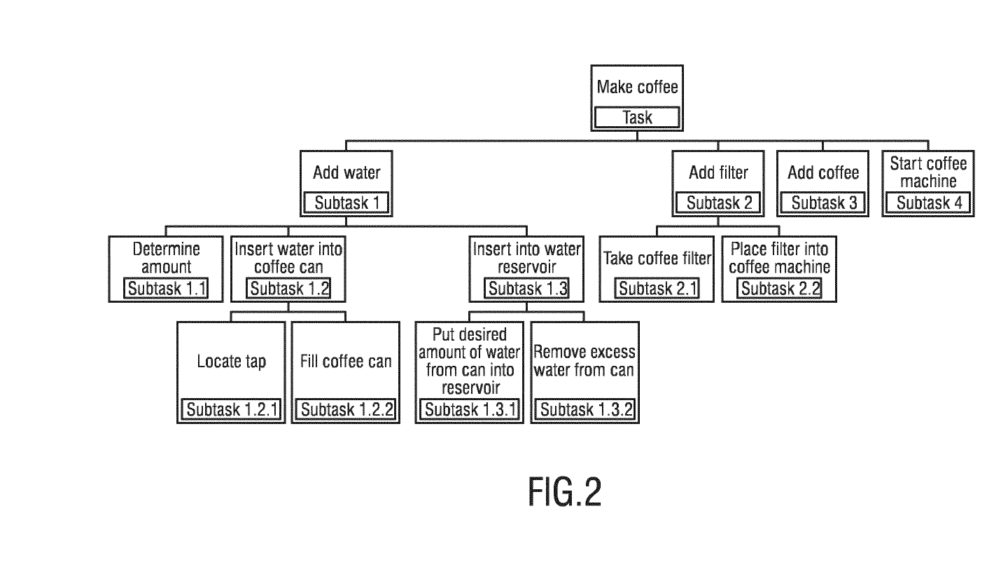

In the following, with reference to the diagram of FIG. 2 illustrating the tasks connected to an exemplary process to be carried out by an appliance suitable for use in the proposed system, the assistance for a cognitively impaired person, e.g. an AD patient, acting in a kitchen environment is described in more detail. This serves as example case for varying skills with respect to the use of kitchen appliances. The task described hereinafter relates to making coffee using a standard drip-filter system 7a as shown in FIG. 1.

It is known that cognitively impaired persons prefer to use appliances 7 that they are used to, i.e. the form-factor and types of actions should be reminiscent of the period before onset of the disease. This use case is added here to show an example of how the data processing and determination of a suitable guidance output to the user can be embodied.

The above described components of the system 10 can be implemented for example as follows:

First, the detection of the patient's status is made to determine at which moment the patient requires additional guidance to help him/her through the task. Patient's status can be based on several features. The most straightforward features can be detected by the appliance itself, such as the time it takes to complete a subtask, or the detection of inappropriate input signals to the device, e.g. pressing the power button of the coffee machine before adding water. Additional features can be based on more extensive monitoring approaches, including methods to detect anxiety, such as camera-based gaze tracking, speech recognition by using a microphone, etc.

If it is detected, e.g. by the appliance 7 itself, in the embodiment according to FIG. 1 the coffee maker 7a, that the user requires additional information or support to complete the task, it is determined which additional information is required. This can be achieved by splitting up the tasks into subtasks, using for example a tree-like structure such as partially displayed in the scheme according to FIG. 2. In FIG. 2, only subtasks 1 and 2 have been subdivided in more detail for visualization purposes.

The coffee maker 7a may detect a problem during the task of making coffee. For example, the user does not appear to be able to add water, because he pushed the start button first. The coffee maker 7a detects the problem: “subtask 1 not completed” and instructs the user itself to add water, for example via a display on the coffee maker 7a or on another display which can be contacted by several other appliances 7. Alternatively, the coffee maker 7a sends the information to the device 1, where the data processing unit generates the adequate assistance data and sends them back to the coffee maker 7a for display.

If this additional instruction is sufficient, the process continues to the next subtask; if not, the coffee maker 7a goes to an additional level of more detailed instructions to guide the user through the subtask “Add water”. By dividing tasks into subtasks using tree-like structures, the user will receive detailed guidance as necessary to complete the task, while the total amount of instructions provided is minimized. Providing needlessly detailed instructions to skilled users would be unnecessarily annoying, thus, only required guidance is provided.

If more than one action is necessary for one subtask, e.g. for “Add water”, blinking light indicators at the relevant items, for example on or in proximity of the coffee can and on a water tap, can be used.

Likewise, the subtask “Add filter” can be guided by flashing a light on the cabinet where the filters are stored and on the coffee maker's 7a filter holder. All other subtasks of the main task “Make coffee” can be broken down to one or more single steps which can be displayed to the user at any point of the process. For example, if adding water works without time delays or wrong actions, the coffee maker 7a will await inserting the filter into the holder next. If the expected action does not take place during a predetermined time, the coffee maker 7a or the device will start assistance.

It should be noted that the device 1 can be arranged as a separate device, e.g. on a computer, smart phone, etc., but may also be integrated into other devices, in particular into a user device or an appliance, such as the coffee maker 7a.

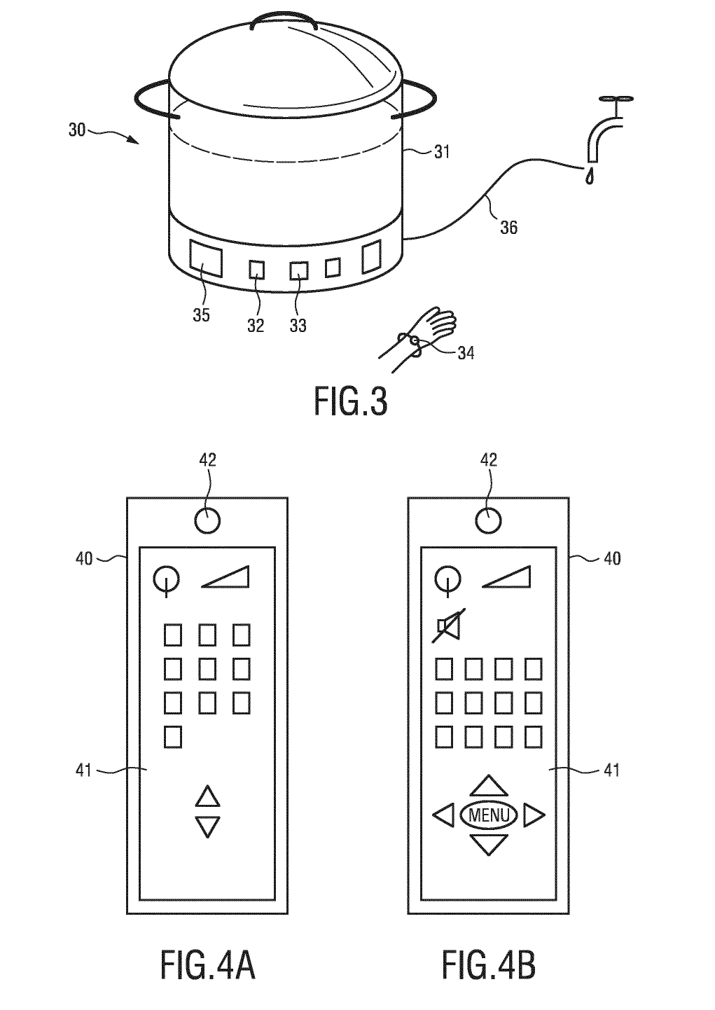

Similarly, the user can be guided through the preparation of a meal, as described hereinafter with reference to FIG. 3 showing a schematic diagram of an exemplary smart cooker representing another embodiment of the device according to the present invention.

A cooking device 30 is in this embodiment a solitary smart cooker 30 which incorporates all necessary components in one body 31. The resulting meal is “the food as wished”, even if the user does not have all required skills for a “normal” cooking procedure.

This is achieved by a change in the functionality of the cooking device 30, wherein certain cooking subtasks are taken over by the cooking device, as to correct the otherwise imperfect procedure because of e.g. the changing condition of the cognitively impaired person.

The integrated guidance system makes use of various actuation methods, such as e.g. auditory or visual signals, as to e.g. remind the user of a next action to be taken. The basics of such methods are known from the art, e.g. the auditory signal of an appliance at the end of a cooking action, but according to the present invention the signals go beyond this simple “end of process signal”. Frequent and/or varying signals are used for intermediate guidance. The trigger for the signals is not only based on the appliance functionality but also on the actual user skills and behaviors. These might include escalation levels of guidance such as e.g. (1) multiple warnings followed by a taking-over or overrule action by the appliance, if certain necessary actions are not carried out by the user, (2) changing the signal method from visual to auditory or mechanical, e.g. with the use of a connected wrist watch with vibration functionality, (3) actuating a signal to e.g. a next room, if the user would be in that next room, based on an available connected/smart/demotic equipped house system.

For example, when the smart cooker 30 is turned on, a finger print sensor 32 on the on/off button of the body 31 identifies the cognitively impaired person as the user of the appliance. A controller 33 connects to e.g. a smart watch 34 of the cognitively impaired person to monitor the anxiety status of the user via heart rate and skin conductance response.

The user now selects a dish to be prepared on a display of the smart cooker 30. As the user has been identified as the cognitively impaired person, the smart cooker 30 will check whether the selected dish is appropriate for the time of day, and ask for reconfirmation if the choice does not fit the usual time or preferences of the user. If selection takes long or two choices are made and then cancelled, combined with the detection of increased anxiety in the user, the smart cooker 30 will suggest a limited list of three choices that are appropriate. In case the user is a caregiver there is no check for fit to the usual time or preferences of the user, and suggestions for recipes are displayed only after four cancellations and a list of ten choices are presented.

As the user has been identified as the cognitively impaired person, the smart cooker 30 will then run a cooking protocol/sequence which is adapted based on the real-time monitored skill and alertness level. The smart cooker 30 will be prepared to take over, abort, or alike, if the status of the user changes. The controller 33 now uses the switches and interaction panels of the smart cooker 30, like lid, mixer button, on-off button 32, input buttons next to the display 35 or touchscreen display, to monitor the progress of the task and the actions performed.

Possible adaptations might be:
Changing the functionality of e.g. a keyboard as to reduce the number of accessible and controllable functions by the user at a certain moment. E.g. the caregiver has all options available at all times and is therefore capable of applying variations to the recipe. For the cognitively impaired person, if it is detected that there is significant hesitation in execution of the tasks or increased anxiety, only the button for the action that is scheduled to be done at that time will be active, so variations to the recipe are not possible. However, for a next step, when there is no hesitation or anxiety detected, the system will deduce this is a familiar step and small variations may be allowed.
Reducing the number of subtasks that are presented to the user, as suggested by either a display message or a verbal message: less detailed tasks for less skilled person with intermediate tasks being taken over by the smart cooker 30, more detailed tasks for the higher skilled person which resemble a DIY process.
Increased level of guidance in subtasks. The recipe for the caregiver is “add 200 ml of water”. The recipe for the cognitively impaired person who is detected to be having a good day is “fill a beaker with 200 ml of water. Add the water to the mixing compartment”. If the system has detected the user is not having a good day, or it takes too long for the water to be added, the recipe is “push the button to add water” and the appropriate volume of water is added from a water reservoir in the smart cooker 30 or from a connected water supply 36.
Automation of subtasks. If in the above situation the button is not pushed within an appropriate time or previous subtasks were needed to be done at a high support level or automation level, the smart cooker 30 will automatically add the appropriate volume of water.

The level of support/guidance can also be set, e.g. via the user panel, in case the cooker operates at a too complex or too simple level according to the user.

The cooker may also be equipped by more elaborate sensors to a more sophisticated monitoring of the user and the actions taken.

A microphone might be added so that the user might set the requested level of guidance/support by a verbal command. In addition, sounds or words from the user can then be used as an input for the user's anxiety level, or as input how well the user understands and copes with the current subtasks. Ambient sounds can be used as activity monitor. E.g. sound of running water, cabinet doors opening and closing, etc. will be satisfactory to keep the process running to the next task.

Cameras in the smart cooker 30 towards the mixing/cooking compartment can be used as activity monitor, e.g. whether ingredient has been added, and cameras aimed towards the front of the smart cooker 30 and/or the user can be used as user monitor for facial expression, gestures, whether operation of the smart cooker 30 is purposeful or hesitant.

In yet another embodiment (not shown), not only the cooker 30 is smart, but it also connects to other smart devices and/or sensors in the kitchen.

This can be used for monitoring the user, such as cameras in other locations in the kitchen to assess facial expression of the user, or remote PPG cameras to assess heart rate of the user. Also the time between opening and closing of a refrigerator door or a cabinet is input for the cognitive status of the user, as well as the number of cabinets that are opened successively indicates a searching pattern. The length and success of it indicates user clarity, skill or familiarity with the kitchen.

Also more elaborate activity monitoring is possible this way, such as monitoring whether the faucet has been running to determine whether a subtask to take water has been performed, or monitoring whether the correct cabinet is opened when an ingredient or bowl has to be taken from that cabinet.

Moreover, also other devices in the kitchen can be actuators. If the user is determined to be unskilled, unfamiliar with the kitchen, or a cognitively impaired person struggling with the task, guidance and support can be offered by the complete kitchen. For instance, at medium level of support spotlights or LED lights indicate the proper cabinet for the utensil or ingredient that is required at that time. Also, if a volume of water is to be taken, at medium level of support/guidance first the cabinet with the beakers is highlighted, and after a beaker has been taken the faucet is highlighted. At a higher level not only the faucet is highlighted but also a short spurt of water is generated to remind the cognitively impaired person of the function of the faucet. At an even higher level the cooker instructs the faucet to dispense the required amount of water when the beaker is detected.

It is clear that monitoring methods that are not embedded in the appliance system concerned will be linked to and be part of an overall connected system 10 as described at the beginning with reference to FIG. 1. As mentioned earlier, this functionality might be provided by the connected system 10.

In the most elaborate embodiment, data from sensors all over the house is used. In this case the assessment of the current cognitive status of the user also takes into account the information from monitoring the user prior to entering the kitchen, such as daily function (feeding, dressing, bathing, etc.) observation and the trends in these daily functions by vision systems (as part of a smart/connected house system).

Adding a second person, e.g. a caregiver in the case of an AD patient, is a next extension. Taking a second person in the loop of the adaptable user interface would include a non-machine based monitoring and actuating function in the system.

The caregiver might e.g. be equipped with an additional connected wireless device, e.g. wrist-watch type, that would allow to remotely monitor both the user and the user's actions through a connection with the appliance(s) in use by the user. The caregiver can then:
add input on the user cognitive status by user monitoring,
add input on the interaction of the user with the appliances and execution of the tasks by activity monitoring,
suggest or override the computed cognitive/skill level of the user by data processing,
be instructed by the system 10 to help the user with certain subtasks by actuation, on-site or remote via video or audio link, or
approve the use of selected appliances, approve deviations from standard protocol, determine to shut down appliances or abort the activity by actuation, on-site or remote.

Next to the quite relevant advantage of being able to prevent mistakes and dangerous situations, the second interfacing by the second person might also allow small corrections and guidance that are not observed by the user but allow him/her to proceed, offering the envisioned pleasure and satisfaction of having positively executed a task. Optionally the second person might join the user in his/her activity, to give personal support and guidance.

Yet another embodiment of a device according to the present invention in the form of a TV remote control 40 with a touchscreen 41 and a camera 42 is described with reference to FIG. 4A and 4B. If not used, the touchscreen 41 shows only the buttons available to the cognitive impaired person, as determined by the caregiver. This can be e.g.: On, Off, Volume up, Volume down, ten favorite TV channels which are displayed as tiles showing the logos of the channels, Channel up, Channel down, as shown in FIG. 4A.

Picking up of the remote control 40 is detected by accelerometers. This triggers the built-in camera 42, which searches for the face of the user. If the device identifies the user as the cognitive impaired person, it remains in the limited state. If the device detects the caregiver or another person who is not the cognitive impaired person, it immediately switches to full controls (as depicted in FIG. 4B), so that normal use is not delayed for the non-cognitive impaired persons.

Based on the detected cognitive skill level, the remote control 40 can have slightly different functionalities. For example, the rate of change of the sound volume can be adjusted according to the cognitive level, and the maximum sound level may be adjusted to the detected cognitive level (e.g. max. set to 50% for low cognitive levels). Another, perhaps more interesting option is to adjust the functionality of the “Channel up/down” button, such that the TV automatically selects a channel that is best suited to the detected cognitive level of the user.

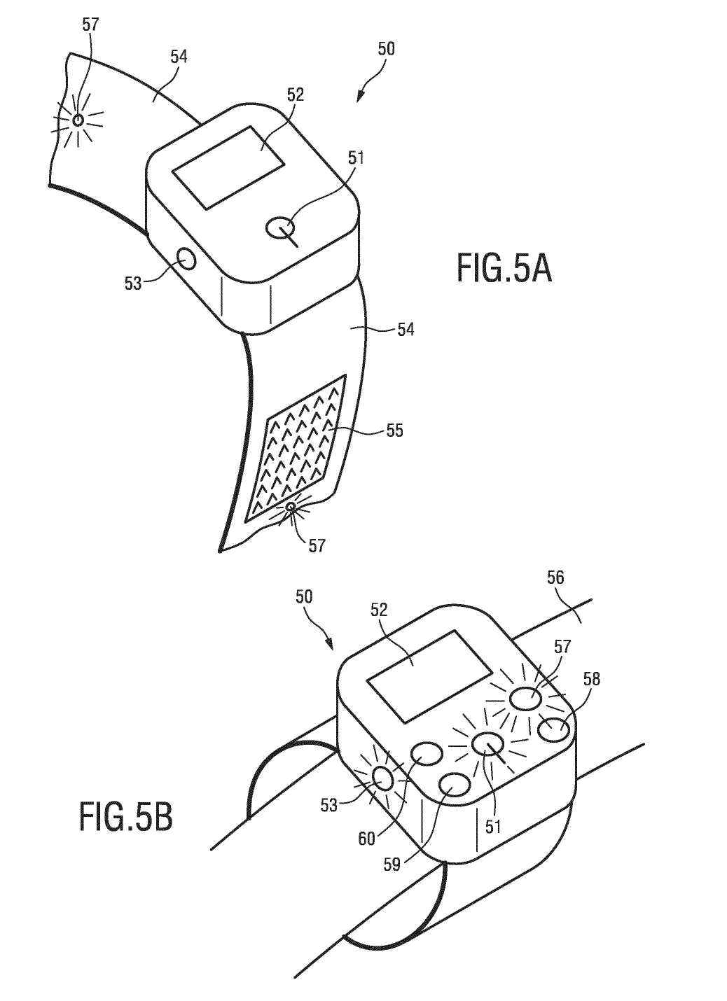

FIGS. 5A and 5B show a further embodiment of the invention referring to a blood pressure gauge 50. Like the remote control 40 of the previous embodiment, the blood pressure gauge can be equipped to adapt its functionality depending on the abilities or the mental state of the user.

In FIG. 5A, the gauge 50 is turned off with only an On-Off-button 51 and a display 52 visible. A speaker 53 can be arranged in the housing of the gauge 50. Gauges 50 like this are usually equipped with two straps 54 with a fastening device like velcro 55 for easy fastening around the wrist 56 of a person. The straps 54 are also suitable to be inflated to work as pressure sleeve for the measurement of the blood pressure.

The gauge 50 can be equipped with an internal watch and/or timer which can be programmed by a caregiver or by another competent person to remind the cognitively impaired person to use the gauge 50 regularly. For example, the speaker can emit a warning signal or a spoken message or the display 52 can show a message accompanied by an audio signal. Based on the reaction of the cognitively impaired person the gauge 50 then can activate further functions and/or signals to guide the person through the process of using the gauge 50.

If e.g. the person is unable to fix the gauge 50 to the wrist 56, LEDs 57 on the straps 54 can e.g. flash to hint what is to be done or a corresponding instruction can be displayed on the display 52. If the cognitively impaired person still is not able to use the gauge 50 or does not know what to do with the straps 54, the gauge 50 may (e.g. automatically) alert the caregiver.

If the gauge 50 is placed correctly, further functions can appear on the housing as shown in FIG. 5B. In a first step, the On-Off-button 51 flashes and/or a message like “Press button” is given by the speaker 53 or via the display 52. Depending on the time the cognitively impaired person needs to follow the instruction to push the button 51, the next instruction will be given or again an alert may be sent to the caregiver.

If the button is pushed in a given time interval, a Start-Button 57 may flash to indicate the next action. The measurement may then take place by inflating the straps 54 and slowly deflating to measure the systolic and diastolic pressure values and the pulse. Further measurements regarding the skin temperature or the conductivity of the skin indicating sweating can be carried out simultaneously if the gauge 50 is equipped with the respective sensors.

In case the gauge 50 detects a rising pulse or skin conductivity during measurement, indicating that the cognitively impaired person feels anxious due to the pressure on the wrist 56, the sound of the inflating straps or generally due to disorientation, a spoken message can be emitted by the speaker, trying to calm down the patient. If this has no effect, again the caregiver can be alerted.

If the cognitively impaired person remains calm and the measurement can take place without complications, the gauge 50 can display the values in the display 52 and then store in an internal storage device or send it to the central device 10 present in the household of the cognitively impaired person. Further embodiments can provide information distribution to a doctor, to the caregiver or other persons in charge.

Other functions can be enabled when the measurement is completed, e.g. a button 58 to alert the caregiver, a button 59 to store the values and a button 60 to show e.g. previous values.

If the cognitively impaired person does not remove the gauge 50 after the measurement, there can be another alert by the speaker 53 or by a flashing LED. If this has no effect, the LEDs on the straps 54 can flash to remind the patient where to start to remove the gauge 50. If the gauge 50 is still in place after a certain time, again an alert can be sent to the caregiver to look after the patient and remove the gauge 50 for the convenience of the patient and to prevent damage to the gauge 50.

In another example for reduced functionality (not shown), a microwave may adjust its maximum power and/or operation time to the user's cognitive levels. For example, the microwave max heating time is restricted to 1 min to prevent food or drinks becoming too hot. In the case of a gas stove, the gas supply is automatically interrupted after, e.g., 20 minutes.

Whereas a remote control 40 for TV operation is easily recognized to the current generation of cognitive impaired persons, this will not be intuitive for many other appliances. For a microwave oven for instance, the control panel needs to be on the device itself In this case, the control panel shows a limited number of controls such as: Heating level high, Heating level medium, Heating level low, Time 10 sec, Time 1 min, Start, Stop.

User identification can in this case e.g. be done by fingerprint scanner on the start and stop buttons. If a user other than the cognitive impaired person is detected, the control panel switches to full controls including setting wattage at more levels, defrosting settings, etc.

A similarly adapted user interface and functionality can be envisioned for a telephone, in which for the cognitive impaired person the control panel displays only on, off, and tiles showing the pictures of a limited number of preferred contacts, whereas upon the detection of any other person the normal phone interface is displayed. Alternatively, as already used in telephones for elderly, the button sizes might be adapted by making them visually larger for the cognitive impaired person. The functionality of the phone might be limited to e.g. basic calls only, but disabling the SMS and internet access functionalities for the less skilled and/or cognitive impaired person. Such functions would most probably not be envisioned by the cognitive impaired person but by wrong use and/or mistake might be activated by the cognitive impaired person, leading to anxiety and because of the anxiety resulting in even loss of the basic envisioned and wanted phone call functionality as well.

In yet another embodiment, the adaptable appliances are connected to a central control unit so that the caregiver can adjust the level of user interface/control panel and/or device functionality that is available to the cognitive impaired person. This way the adaptations can be customized to the cognitive level of the cognitive impaired person, and the caregiver can compensate for further cognitive decline.

In addition, this central control unit can also provide information to the caregiver on the interaction of the cognitive impaired person with the devices. This may help the caregiver to set the level of the interaction and functionality, and to track the current cognitive level and trends.

The control unit may also link to other sensors, cameras and smart devices that are present in the house. These can provide input for user recognition, e.g. a camera or microphone in the kitchen or living room, and user monitoring, e.g. anxiety levels via heart rate and galvanic skin response measured by a smart watch. Vice versa, the interaction with the user interface/control panel and the user recognition of the device/appliance can be used as input for the smart home system for tracking of presence of the cognitive impaired person and activity patterns.

Furthermore, the system may be enhanced by combining it with functions regarding the caregiver. If the system detects use by the cognitive impaired person, but it also detects the presence of a recognized caregiver in the same room, e.g. via other monitoring or recognition devices, the functionality of the device/appliance is not restricted. In contrast, the user interface/control panel remains in the status for the cognitive impaired person as it would be very confusing for them to work with two different user interfaces/control panels.

Additionally, the system could also inform the caregiver of use of the device/appliance, and allow the caregiver to shut down the device/appliance if needed. This alarming could be set at different levels, e.g. at every use, if potentially dangerous/harmful settings are selected, or if the interaction of the cognitive impaired person with the user interface/control panel is erratic indicating an episode of anxiety or panic.

The system may also monitor the user to determine whether and to which extend functionality of the device should be restricted. Similar to previous embodiments it would be counter-productive to have a dynamic adaptation of the user interface/control panel as this would confuse the cognitive impaired person. Input for determining the current mental state of the cognitive impaired user are analysis of the use of the user interface/control panel, facial recognition if the device/appliance is fitted with a camera, or other appropriate methods involving sensors on the device/appliance such as those used for user recognition, or other sensors, cameras and smart devices in the room/house as mentioned above.

Assuming that devices in use are connected, the caregiver can be notified immediately about a certain device becoming activated. Alternatively, to prevent caregiver from receiving non-essential notifications, these notifications can be at the moments when dangerous or non-desired use is detected.

For example, when the cognitively impaired person starts using the oven, the oven will communicate this to the smart phone of the caregiver. However, no visible notification will be produced. The software running in the smart phone will be evaluating the use of the oven. Once a certain event is triggered, for example, oven has been on for more than 10 min, and its door has not been opened, then a visible notification will be generated indicating that there may be erroneous use of the oven.

In the aforementioned embodiments, mainly the case of a cognitive impaired person suffering from Alzheimer Disease or other forms of dementia in an early phase of the disease has been described. The user performs kitchen tasks like cooking in a home environment. The suggested methods and embodiments are, however, equally well relevant for any other situations or circumstances or persons (patients), where there is a need or advantage in having adaptable user guidance and adaptations to devices towards varying skill level for persons performing a task. This can be in kitchen, bathroom or other home or workplace environments. User can be various persons and not only patients suffering from a disease, such as elderly people that are another obvious target user group, unskilled people, or people in an unfamiliar environment (such as a holiday apartment).

A processor or a processing unit is one example of a controller which employs one or more microprocessors that may be programmed using software (e.g., microcode) to perform the required functions. A controller may however be implemented with or without employing a processor, and also may be implemented as a combination of dedicated hardware to perform some functions and a processor (e.g., one or more programmed microprocessors and associated circuitry) to perform other functions.

Computer program code for carrying out the methods of the present invention by execution on the processing unit 3 may be written in any combination of one or more programming languages, including an object oriented programming language such as Java, Smalltalk, C++ or the like and conventional procedural programming languages, such as the “C” programming language or similar programming languages. The program code may execute entirely on the processing unit 3 as a stand-alone software package, e.g. an app, or may be executed partly on the processing unit 3 and partly on a remote server. In the latter scenario, the remote server may be connected to the head-mountable computing device through any type of network, including a local area network (LAN) or a wide area network (WAN), or the connection may be made to an external computer, e.g. through the Internet using an Internet Service Provider.

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions to be executed in whole or in part on the processing unit 3, such that the instructions create means for implementing the functions/acts specified in the flowchart and/or block diagram block or blocks. These computer program instructions may also be stored in a computer-readable medium that can direct the cardiopulmonary resuscitation guidance system including the portable computing device to function in a particular manner.

The computer program instructions may, for example, be loaded onto the portable computing device to cause a series of operational steps to be performed on the portable computing device and/or the server, to produce a computer-implemented process such that the instructions which execute on the portable computing device and/or the server provide processes for implementing the functions/acts specified in the flowchart and/or block diagram block or blocks. The computer program product may form part of a patient monitoring system including a portable computing device.

Examples of controller components that may be employed in various embodiments of the present disclosure include, but are not limited to, conventional microprocessors, application specific integrated circuits (ASICs), and field-programmable gate arrays (FPGAs).

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. A device for providing assistance to a person using an appliance, said device comprising:
a data input for obtaining person related data representing data obtained by monitoring the person,
a data processing unit for processing the obtained person related data to:
determine the person's mental state and/or cognitive level from the obtained person related data,
detect if the person has limited or no ability to perform a desired task, wherein the desired task pertains to the usage of the appliance by the person, and
generate assistance data in case of detection that the person has limited or no ability to perform the desired task and/or if the person's current mental state and/or cognitive level is below a threshold or out of a distribution or value range required to perform the desired task, said assistance data representing data directed to supporting the person in performing the desired task, wherein said assistance data are used to adapt the functionality of the appliance by adapting the number of functions accessible and controllable by the person for said appliance according to the person's current mental state and/or cognitive level,
adjust the assistance data in dependence of the performance of the person in carrying out the desired task when i) the threshold value required for the desired task is exceeded or not, accomplished, or ii) when the distribution or value range is exceeded or not accomplished, and
a data output for providing said assistance data for supporting the person in performing the desired task, and for adapting the functionality of the appliance based on said assistance data.

2. A device according to claim 1,
further comprising a recognition unit for recognizing the person's identity, wherein the data processing unit is configured to take the person's identity into account in the generation of the assistance data.

3. A device according to claim 1,
wherein the assistance data are control data inducing the start, execution and/or end of a task on an appliance, the assistance data being transmitted to the appliance on which the task is being carried out when the data processing unit has detected that the person's ability to perform the task is below the threshold or out of the distribution or value range required to perform said task.

4. A device according to claim 1,
wherein the assistance data include instructions how to carry out a task on an appliance, the assistance data being communicated to the person carrying out the task depending on the person's current mental state and/or cognitive level when the data processing unit has detected that the person's ability to perform the task is below the threshold or out of the distribution or value range required to perform said task.

5. A device according to claim 1,
wherein the person related data are obtained by monitoring the person by way of a monitoring unit, the person related data being one or more of: heart rate, pulse, blood pressure, sweat, skin and/or core body temperature, facial expression, gaze track, pupil size, posture, motion, trembling, repetitive movements, tics, twitches, voice, tactile abilities, amount of reaction to outer stimuli, reaction time to outer stimuli.

6. A device according to claim 5,
wherein the data processing unit is configured to determine the person's current mental state and/or cognitive level from the monitored data.

7. A device according to claim 6,
wherein the data processing unit is further configured to compare current person related data indicating the mental state and/or cognitive level to predetermined person related data and/or person related data from previous monitoring of the person and/or threshold values and/or distribution or other value ranges assigned to tasks and generating assistance data depending on the result of the comparison.

8. (canceled)

9. A device according to claim 1,
further comprising a data storage to store the person related data, data obtained from the data input, the assistance data, threshold data, distribution or other value range data and/or comparative data derived from previous monitoring of the person.

10. A system for providing assistance to a person using an appliance, said system comprising:
a monitoring unit for monitoring the person and generating person related data,
a device as claimed in claim 1 for generating assistance data based on the person related data obtained from the monitoring unit, and
a user device for obtaining and/or using said assistance data to support the person in performing the desired task.

11. A system according to claim 10,
wherein the monitoring unit comprises one or more of a tactile recognition device, in particular a fingerprint scanner, a device for recognition and/or analysis of voice or speech characteristics, preferably a microphone, a facial recognition or facial expression device, a device for gaze tracking, a device for posture recognition or analysis, preferably a camera, a device for monitoring vital functions, preferably a pulse and/or heart rate and/or blood pressure and/or skin or body core temperature monitor, a device for detecting amount of response and reaction time to outer stimuli, in particular a timer, or an device for reading a personal identification tag.

12. A system according to claim 10,
wherein the user device is configured to communicate instructions to the person carrying out a task on an appliance depending on the assistance data generated by the data processing unit when the data processing unit has detected that the person's ability to perform the task is below the threshold or out of the distribution or value range required to perform said task.

13. A system according to claim 10,
wherein the user device is configured to carry out a task on an appliance automatically when the data processing unit has detected that the person's ability to perform the task is below the threshold or out of the distribution or value range required to perform said task.

14. A method for providing assistance to a person using an appliance, said method comprising:
obtaining person related data representing data obtained by monitoring the person;
processing the obtained person related data to:
determine the person's mental state and/or cognitive level from the obtained person related data,
detect if the person has limited or no ability to perform a desired task, wherein the desired task pertains to the usage of the appliance by the person, and
generate assistance data in case of detection that the person has limited or no ability to perform the desired task and/or if the person's current mental state and/or cognitive level is below a threshold or out of a distribution or value range required to perform the desired task, said assistance data representing data directed to supporting the person in performing the desired task;
adjusting the assistance data in dependence of the performance of the person in carrying out the desired task when i) the threshold value required for the desired task is exceeded or not accomplished, or ii) when the distribution or value range is exceeded or not accomplished.
adapting the functionality of an appliance by adapting the number of functions accessible and controllable by the person for said appliance according to the person's current mental state and/or cognitive level, and
providing said assistance data for supporting the person in performing the desired task.

15. A non-transitory computer readable medium, comprising computer readable code embodied therein, the computer readable code being configured such that, on execution by a suitable computer or processor, the computer or processor is caused to
receive person related data representing data obtained by monitoring the person:
process the obtained person related data to.
determine the person's mental state and/or cognitive level from the obtained person related data,
detect if the person has limited or no ability to perform a desired task. wherein the desired task pertains to the usage of the appliance by the person, and
generate assistance data in case of detection that the person has limited or no ability to perform the desired task and/or if the person's current mental state and/or cognitive level is below a threshold or out of a distribution or value range required to perform the desired task, said assistance data representing, data directed to supporting the person in performing the desired task,
adjust the assistance data in dependence of the performance of the person in carrying out the desired task when i) the threshold value required for the desired task is exceeded or not accomplished, or ii) when the distribution or value range is exceeded or not accomplished;
adapting the functionality of an appliance by adapting the number of functions accessible and controllable by the person for said appliance according to the person's current mental state and/or cognitive level; and
providing said assistance data for supporting the person in performing the desired task.
